(19) United States
(12) Patent Application Publication
(10) Pub. No.: US 2026/0367060 A1
(43) Pub. Date: Nov. 19, 2026

(54) SYSTEM FOR ANALYZING BEVERAGE QUALITY AND FLAVOR PROFILE

(71) Applicant: KARADENIZ TEKNIK UNIVERSITESI TEKNOLOJI TRANSFERI UYGULAMA VE ARASTIRMA MERKEZI MUDURLUGU, Trabzon (TR)

(72) Inventors: Yavuz KABLAN, Trabzon (TR); Ayten ATASOY, Trabzon (TR); Elif BAYKAL KABLAN, Trabzon (TR)

(73) Assignee: KARADENIZ TEKNIK UNIVERSITESI TEKNOLOJI TRANSFERI UYGULAMA VE ARASTIRMA MERKEZI MUDURLUGU, Trabzon (TR)

(21) Appl. No.: 19/603,215

(22) PCT Filed: Nov. 4, 2025
(86) PCT No.: PCT/TR2025/051387

§ 371 Date: Jul. 2, 2026

(30) Foreign Application Priority Data
Nov. 22, 2024 (TR) 2024/016601

(51) Int. Cl. G01N 33/14; G01N 27/12; G01N 33/00

(52) U.S. Cl. CPC G01N 33/14; G01N 27/125; G01N 33/0001; G01N 33/0047

(57) ABSTRACT

A beverage quality and flavor profile analysis system includes an Electronic Nose (E-Nose) device, an Electronic Tongue (E-Tongue) device, and an artificial intelligence infrastructure based on electroencephalography (EEG) signals obtained from individuals who taste and evaluate beverages (particularly tea). When interacting with volatile organic components emitted from the airtight glass container containing the beverage through teflon tubes, the sensors in the E-Nose device produce a conductivity value depending on a type of gas and, through measurements, obtain information on how much volatile organic components each type of beverage emits under same ambient conditions. The E-Tongue device includes electrodes for determining presence and amount of chemical components in the beverage in order to perform voltammetric and amperometric measurements to allow chemical components of the beverage to be analyzed by electroanalytical methods in the airtight glass container containing the beverage.

### TECHNICAL FIELD

The invention relates to a beverage quality and flavor profile analysis system comprising an Electronic Nose (E-Nose), an Electronic Tongue (E-Tongue), and an artificial intelligence infrastructure based on electroencephalography (EEG) signals obtained from individuals who taste and evaluate beverages (particularly tea).

### PRIOR ART

Current practices in tea production offer certain advantages in addressing the complexity of determining tea quality. However, these practices also present some shortcomings and technical challenges.

The evaluation of the tea's flavor profile is traditionally carried out by trained expert tasters through direct tasting methods. This approach, however, suffers from subjectivity and low sensitivity. Differences due to tasters' personal preferences, the reduction or loss of taste and smell abilities due to various illnesses, and the gradual dulling of the palate after long tasting sessions can all hinder accurate assessments. This may result in the production of non-standard products.

Various laboratory tests are also used to analyze the chemical components of tea. However, the high costs and time-consuming processes of these tests unnecessarily lengthen the production process and can make it economically unsustainable. To deal with such challenges, tea producers need low-cost and stable solutions. In response to this need, technological tools such as E-Nose and E-Tongue have been developed. However, these methods do not fully capture the complexity of the human sense of taste and smell in decision-making and are therefore not fully utilized as a standard.

E-Nose analyzes volatile organic compounds in the air, while E-Tongue makes measurements through liquid contact sensors. These devices speed up and automate the process of determining the quality of tea, minimizing human intervention. Furthermore, these methods are inexpensive, do not require complex sample preparation, and provide reproducible results with high accuracy. However, E-Nose and E-Tongue have some shortcomings and technical challenges. For example, these devices may face limitations in detecting certain ingredients and may not offer as broad a flavor profile as human tasters. In addition, these technologies can be costly to develop and maintain.

As a result, despite the advantages of current practices, there are still some technical challenges and shortcomings in the determination of tea quality. Technological solutions such as E-Nose and E-Tongue only measure chemical components and ignore the human sensory response. However, the ability of these technologies to precisely mimic the extensive analytical capability offered by the human senses, with even more detailed analysis of the flavor profile and smart sensors, could ensure that consumers get exactly the quality products they want.

During tastings by tea tasters, data from the three basic senses of trained experts such as sight, smell and taste are used to fully characterize and statistically analyze food products throughout the production process and during storage process. These analyses have been widely used in the field of food technology in recent years for various purposes such as the development of new processes and products, quality control, consumer acceptability, taste and flavor characterization. However, while taste tests and laboratory tests are excellent at distinguishing bad products, they are not very effective at predicting which products will be more attractive to consumers. Therefore, although various sensor technologies have been developed and used for sensory analysis of product categories, the possibility of reliably predicting food choice or purchasing behavior still seems elusive.

In the last decade, there has been a huge surge of interest in the assessment of emotions in consumer and sensory sciences. In particular, the degree to which an emotion is positive or negative is central to sensory science, with the assumption that quality products are more likely to be appreciated. Therefore, the role of emotions in the perception of food quality seems to be extremely important.

Neurophysiological techniques such as EEG, used to measure brain activity, are increasingly used in food research. EEG allows consumers to collect data over long periods of time without discomfort, which allows for more in-depth analysis. The use of EEG in food research has increased significantly in recent years as a tool for understanding psychological and emotional states through the observation of brain waves.

### OBJECT OF THE INVENTION

The invention has been developed to create a flavor perception structure similar to that of humans. It has an artificial intelligence system that comprises an E-Nose, an E-Tongue, and EEG signals obtained from individuals who taste and evaluate tea. The object of this system is to mimic the relationship between flavor perception and preference by utilizing human olfactory, gustatory, and brain signals. In this way, the aim is to maintain the quality of the products currently manufactured in beverage production facilities and organizations, determine whether the flavor profiles of new products align with consumer taste preferences, and improve the flavor of health-oriented medicinal beverages to appeal to consumer palates, thus contributing to flavor-mimicking technologies.

The solution offered within the scope of the invention to eliminate the shortcomings caused by the lack of consideration of consumer taste, resulting from processing the data obtained by E-Nose and E-Tongue devices independently of human senses is the use of E-Nose, E-Tongue and EEG signals of tasters together. This system structure can be created by combining three off-the-shelf systems or it can be assembled in a single device. The benefits of using these three systems together can be listed as follows:
Comprehensive Sensory Analysis: E-Nose and E-Tongue devices make objective measurements of the senses of smell and taste independently of the human senses. EEG, on the other hand, reveals consumers' sensory and psychological responses by monitoring brain waves. This triple combination provides a more comprehensive and holistic assessment in the sensory analysis of food products. The chemical components of the tested tea can be detected by E-Nose and E-Tongue, while the sensory response of the consumer to this beverage can be interpreted using EEG. This provides a clearer link between the sensory features of products and consumer reactions.
Advanced Quality Control: Quality control plays a critical role in food production processes. E-Nose and E-Tongue devices provide accurate and repeatable data regarding the smell and flavor of products. EEG, on the other hand, reveals how these data are perceived and evaluated by consumers. This gives manufacturers a more comprehensive understanding in the quality control phase and increases the likelihood that products will meet consumer expectations.
Sensory and Emotional Compatibility: Consumers' emotional responses to products can determine their success in the market. When emotional data obtained with EEG is combined with E-Nose and E-Tongue data, it is possible to fully understand how food products are perceived both sensory and emotionally. This compatibility can also drive product development processes in a way that increases consumer satisfaction.
Prediction of Consumer Behavior: E-Nose and E-Tongue devices create sensory profiles of products. EEG analyzes consumer brain responses to these sensory profiles. This combined approach provides greater accuracy in predicting which products will be more liked and preferred by consumers. Using this data, manufacturers can optimize their marketing strategies and product portfolios.
Target Audience Analysis and Segmentation: EEG can measure the emotional responses of different consumer groups to food products. When combined with E-Nose and E-Tongue data, this data can be used to identify which types of products different demographic groups, age groups, or geographic regions react more positively to. This enables products to be identified and marketed more effectively.
New Product Development: The combined use of sensory and emotional analysis in the development of new food products enables the creation of innovative products that are more in line with consumer expectations. E-Nose and E-Tongue assess the sensory features of new flavor and aroma combinations, while EEG measures the emotional impact of these combinations on consumers. In this way, more market-oriented and consumer-friendly products can be developed.

As a result, the combined use of E-Nose, E-Tongue, and EEG signals enables deeper analysis of food products from both sensory and emotional perspectives. In addition to increasing consumer satisfaction, this approach offers great advantages in product development and marketing strategies. As a new way of understanding food technology and consumer tastes, the integration of this triple system can contribute to bringing more successful and desirable food products to market in the future.

### DETAILED DESCRIPTION OF THE INVENTION

For a better understanding of the definition of the invention, an example of tea as a beverage was used. However, the invention can be used for all types of beverages, including tea. In this context, the invention relates to a system consisting of an E-Nose device, an E-Tongue device, an EEG recorder, an electronic device including software, an airtight glass container, and teflon tube parts. The E-nose device is used to obtain and analyze the conductivity changes caused by the volatile organic components emitted from the beverage on the gas sensors housed in the device. The E-Tongue device is used to analyze the electrical changes of the brewed beverage on different types of electrodes (sensors) housed in the device, using voltammetric and amperometric measurements, and to determine the chemical components. The EEG recorder is used to record the emotional and sensory signals that occur in the brain of the taster as they taste the beverage. Computers are used to analyze and classify a wide range of data obtained from E-Nose, E-Tongue, and EEG devices. The airtight glass container is used to hold the brewed tea so that the E-Nose, E-Tongue and EEG measurement devices can perform analysis together. The teflon tube is used to transfer the beverage odor containing volatile organic components from the glass container to the E-Nose chamber.

The invention makes it possible to reach an absolute result by combining the analysis results obtained by using three separate systems independently. The functions of these systems are described below.
1. The E-nose device (1) performs various analyses by taking volatile organic components emitted from the beverage contained in an airtight glass container (5) through teflon tubes (6). These analyses are based on the effect of volatile organic compounds on the sensors. When each sensor interacts with a specific type of gas, it produces a conductivity value depending on the type of gas it is measuring. The E-nose device has multiple sensors and each sensor makes conductivity measurements specific to certain gases. These measurements provide information on how much volatile organic components each type of beverage emits under the same ambient conditions. This provides a better understanding of the differences in volatile organic components between different beverage types.
2. The E-Tongue device (2) performs voltammetric and amperometric measurements using electrodes (7) in an airtight glass container (5) containing the beverage. These measurements allow the chemical components of the beverage to be analyzed by electroanalytical methods. The E-Tongue device is an electroanalytical device capable of making voltammetric and amperometric measurements on liquids. The E-Tongue device has multiple sensors and each sensor makes measurements specific to different chemical compounds. These sensors are customized to determine the presence and amount of different chemical components in the beverage. Each sensor reacts to a specific chemical compound, making voltage or current measurements that provide detailed information regarding the chemical profile of the beverage. This allows the differences between beverage types to be analyzed more precisely and provides detailed data on how the chemical components of each beverage vary.

To determine the quality and adequacy of the beverage, the brain signals of the taster are recorded with an EEG recorder (3). The EEG device precisely records electrical activity in different areas of the brain during tasting, allowing to analyze the effects of the sensory features of the beverage on the brain. In this process, EEG provides objective data, especially regarding the taster's sensory perceptions (e.g. bitterness, sweetness, aromatic intensity) and emotional responses (e.g. satisfaction, relaxation, enjoyment). When the taster tastes the beverage, activity in areas of the brain associated with taste, smell, and emotional responses (e.g. the orbitofrontal cortex, insula and amygdala) is monitored with an EEG device. These analyses help to establish a neurological “signature” for the sensory quality of the beverage. The distinctive aspect of EEG in this study is that it goes beyond the subjective evaluations of the taster and shows how the sensory effects of the beverage are perceived directly in the brain. In particular, comparing the effects of beverages of different quality levels on the brain provides a deeper understanding of the differences between beverage types and quality levels. These data can be used to correlate volatile organic components (analyzed by E-Nose) and chemical components (analyzed by E-Tongue) of the beverage with brain responses.

Combination of Methods: The analysis of the quality and flavor profile of the beverage includes the processing and classification stages of data obtained from three different devices (E-Nose, E-Tongue, and EEG). The signals obtained in this process may contain various artifacts due to experimental settings, ambient noise, or biological effects. Data preprocessing methods are applied to minimize the distorting effects of these artifacts. For data classification, statistical and frequency-based features are calculated to extract meaningful information from the signals. The data obtained in the feature extraction phase is analyzed with machine learning based algorithms for quality classification of the beverage. The use of this invention enables the rapid implementation of sensory analysis, quality control, consumer preference level assessment, sensory and emotional compatibility, consumer behavior prediction, target audience analysis and segmentation, and new product development processes.

What is claimed is:

1. A system for performing beverage quality and flavor profile analysis, comprising:
an E-Nose device,
an E-Tongue device,
an electroencephalography (EEG) recorder configured to record brain signals of a taster to determine quality and adequacy of a beverage,
an electronic device containing software configured to perform processes of pre-processing, feature extraction and classification of data obtained from the E-Nose device, the E-Tongue device and the EEG recorder,
an airtight glass container containing the beverage to be analyzed, and
teflon tube parts;
wherein the E-Nose device comprises sensors, wherein the sensors, when interacting with volatile organic components emitted from the airtight glass container containing the beverage through teflon tubes, produce a conductivity value depending on a type of gas measured by the sensors and, through measurements, obtain information on how much volatile organic components each type of beverage emits under same ambient conditions, and
the E-Tongue device comprises electrodes for determining presence and amount of chemical components in the beverage in order to perform voltammetric and amperometric measurements to allow chemical components of the beverage to be analyzed by electroanalytical methods in the airtight glass container containing the beverage.

2. The system according to claim 1, wherein the beverage is tea.
